SEQUENCE LISTING (5 sequences)

SEQ ID NO: 1 - protein, 590 aa, Artificial Sequence [sequence omitted]

SEQ ID NO: 2 - protein, 312 aa, Artificial Sequence
  scFv-MT2 Fusion Protein
LVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGGMDPNCSCAAGDSCTCAGSCKCKECKCTSCKKSCCSCCPVGCAKCAQGCICKGASDKCSCCAPGSAGGSGGDSMAEVQLLE

SEQ ID NO: 3 - protein, 182 aa, Artificial Sequence
  Ferritin Heavy Chain
MTTASTSQVRQNYHQDSEAAINRQINLELYASYVYLSMSYYFDRDDVALKNFAKYFLHQSHEEREHAKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 4 - protein, 175 aa, Artificial Sequence
  Ferritin Light Chain
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 5 - protein, 80 aa, Artificial Sequence
  MT2
MDPNCSCAAGDSCTCAGSCKCKECKCTSCKKSCCSCCPVGCAKCAQGCICKGASDKCSCCAPGSAGGSGGDSMAEVQLLE

(19) United States
(12) Patent Application Publication
(10) Pub. No.: US 2011/0200524 A1
(43) Pub. Date: Aug. 18, 2011

(54) VECTOR FOR USE IN MEDICINE

(75) Inventors: Mairi Robertson, Glasgow (GB); David Pritchard, Dundee (GB); Prabhjyot Dehal, Dundee (GB); Claire Geekie, Dundee (GB)

(73) Assignee: ITI SCOTLAND LIMITED, Glasgow, GB (GB)

(21) Appl. No.: 12/990,425

(22) PCT Filed: May 1, 2009
(86) PCT No.: PCT/EP2009/055321

§ 371 Date: Mar. 3, 2011

(30) Foreign Application Priority Data
May 2, 2008 (GB) 0808090.5

(51) Int. Cl. A61K 51/00; A61P 7/02; A61K 38/57; A61K 39/395; A61P 35/00; A61M 1/00

(52) U.S. Cl. 424/1.11; 514/14.2; 424/178.1; 604/28

(57) ABSTRACT

Provided is a vector for use in delivering a therapy to or removing a therapy from a site within a patient, wherein the vector comprises: a) a binding moiety; and b) a therapeutic agent, wherein the binding moiety comprises a metal-binding protein, polypeptide or peptide which is bound to or encapsulates a magnetic or magnetisable substance, and wherein the vector is to be administered using a device comprising an electromagnet and an element suitable for bringing the therapeutic agent into proximity with the site.

### FIELD OF THE INVENTION

The present invention concerns the use of magnetic proteins, peptides and polypeptides in medicine. The uses have significant advantages in that they enable targeting of therapeutic agents utilising the magnetic proteins to specific areas of the body. The presence of the magnetic substance allows more sophisticated spatial manipulation of the therapy within the patient, which is particularly beneficial for therapeutic agents that have an effect on both healthy and diseased tissue and cells. As well as causing localisation of therapeutic agents at the target site, the use of the magnetic proteins according to the invention also enables restraint and control of the therapeutic agent, and even allows for the agent's removal. Still further, the invention relates to medical uses wherein the magnetisable substance is itself capable of causing the therapeutic effect. The invention also concerns products, such as pharmaceutical compositions, and methods relating to the medical use.

### BACKGROUND OF THE INVENTION

The targeting of therapeutic agents to specific sites within the human/animal body is known in the prior art. An example of targeted therapy is the controlled delivery of radiotherapeutic agents to cancerous tissues using antibodies that are radiolabelled. The antibody, in binding to its antigen, delivers the radio-isotope attached to it to the desired target.

Examples of radiolabelled antibodies currently approved for clinical use in the United States for non-Hodgkin's lymphoma include: ⁹⁰Y Zevalin (also known as ibritumomab or tiuxetan) (Biogen Idec Inc.) and Bexxar (also known as ¹³¹I-tositumomab) (GlaxoSmithKline). An example of an antibody conjugated to a drug approved for use in the United States and the UK is Mylotarg (also known as gemtuzumab or ozogamicin) (Wyeth).

One of the advantages of such a targeting approach is that it minimises the impact of a harmful therapeutic agent on healthy tissue and cells.

The binding and subsequent cross linking of receptors on a cell surface by a therapeutic agent may be sufficient to destroy that cell or establish antagonistic signalling responses that may dampen the cells response (e.g. down-regulating an immune response). Many antibodies approved for use in the United States are effective because they work in this way. Examples include: ReoPro (also known as abciximab) (Centocor, Inc.) which is used in the prevention of platelet mediated clots in coronary angioplasty, and Zenapax (also known as daclizumab) (F. Hoffman-LaRoche Ltd) and Simulect (also known as basiliximab) (Novartis AG) which are used to for prophylaxis of acute kidney-transplant rejection.

Many antibodies also eliminate cells by antibody directed cellular cytotoxicity (ADCC) or complement directed cytotoxicity (CDC). It has been suggested that patients with polymorphisms in their Fc gamma receptors (e.g. Fc gamma receptor IIa and IIIa) may be less able to elicit an effective ADCC response as the antibodies do not bind the receptors as efficiently. Alternative therapeutic approaches using targeting therefore may be advantageous in these patients.

Other targeted approaches known in the art are those utilised by gene therapy in which the therapeutic agent is a gene delivered to a cell in an engineered viral particle. This approach makes use of the natural affinity of the viral capsid proteins for the receptors on the surface of a cell, which in normal circumstances enable the wild-type virus to infect specific cell types. As with radio-labelled antibodies the binding of a target cell by the engineered viral particle carrying the therapeutic gene enables the gene to be delivered to specific cell types.

Alternatively, treatments can make use of small molecule drugs, such as Tamoxifen, which themselves are able to bind to hormone (or other) receptors on the surface of specific cell types, thereby blocking the binding of the natural ligand.

Target therapeutic agents now form an integral part of human and animal medicine. However there is an ongoing need to provide different targeting methods which provide alternative or additional advantages to the use of antibodies or ligands to target specific cells or tissue within a body.

It is the aim of the present invention to address this need and to provide an alternative delivery or targeting system that can be used to direct therapeutic agents to specific areas, tissues or cells of the human or animal body.

### SUMMARY OF THE INVENTION

Accordingly the present invention provides a vector for use in delivering a therapy to or removing a therapy from a site within a patient, wherein the vector comprises:
a) a binding moiety; and
b) a therapeutic agent,
   wherein the binding moiety comprises a metal-binding protein, polypeptide or peptide which is bound to or encapsulates a magnetic or magnetisable substance,
   and wherein the vector is to be administered using a device comprising an electromagnet and an element suitable for bringing the therapeutic agent into proximity with the site.

The inventors have determined that it is possible to use a magnetic protein, polypeptide or peptide as a vector or carrier for a therapeutic agent. Further the inventors have surprisingly discovered that it is possible to use such a vector to localise the therapeutic agent to specific areas within a patient's body as well as to remove the vector. This ability is based on the vector's magnetic properties. The vector can be manipulated because it incorporates a magnetic or magnetisable substance, which responds to a magnetic field. In particular, the vector comprises metal atoms or ions (or compounds containing them) in a metal-binding protein, polypeptide, or peptide.

As with other targeted approaches the use of such a vector enables the localisation of potentially harmful therapeutics only to the area that needs to be treated. This also leads to the more efficient use of therapeutic agents; as the agent can be localised the dose need only be sufficient to achieve a therapeutic concentration at the target site, as opposed to having to achieve this dose systemically.

Unlike other targeted approaches, however, the ability of the present invention to remove the therapeutic agent provides additional advantages. In particular, the therapeutic agent can be used at higher doses for short time periods, without the increased side effects frequently encountered with high dose agents. Still further, the ability to remove the therapeutic agent from the body allows treatment using highly active agents without the risks associated with long term exposure to the agent which would otherwise preclude their use.

In addition, the presence of a magnetic element within the vector allows it to be visualised after it has been administered to the patient. This allows the location of the vector within the patient's body, and therefore the location of the therapeutic agent, to be determined. For example, this can be done using MRI scanning.

A further advantage is that the magnetic properties of the vector facilitates its production and purification. In particular, the vectors comprising the therapeutic agent are simple to purify using established techniques, such as affinity purification, or magnetic field purification. The delivery vectors of the present invention have the further advantage that they may be magnetised or de-magnetised using simple chemical procedures.

The present inventors have also discovered pharmaceutical compositions that are particularly preferred for use in the present invention. In particular the pharmaceutical composition according to the present invention comprises (a) a binding moiety; (b) a therapeutic agent; and (c) a recognition moiety, wherein the binding moiety comprises a metal-binding protein, polypeptide or peptide which is bound to or encapsulates a magnetic or magnetisable substance.

In one embodiment of this aspect of the present invention the magnetic or magnetisable substance itself is the therapeutic agent and is used in conjunction with treating the patient with a magnetic field. In particular the pharmaceutical composition is for use in treating diseased cells or tissues in a patient susceptible to hyperthermia, wherein the composition is to be administered prior to exposing the patient to an electromagnetic field.

In a further aspect of the present invention the pharmaceutical composition comprises a therapeutic agent in addition to the magnetic or magnetisable substance, the therapeutic agent being attached to the binding moiety.

In a still further aspect, the present invention provides the use of a vector for the manufacture of a medicament for use in delivering a therapy to or removing a therapy from a site within a patient, wherein the vector comprises:
a) a binding moiety; and
b) a therapeutic agent,
   wherein the binding moiety comprises a metal-binding protein, polypeptide or peptide which is bound to or encapsulates a magnetic or magnetisable substance,
   and wherein the vector is to be administered using a device comprising an electromagnet and an element suitable for bringing the therapeutic agent into proximity with the site.

An additional aspect of the present invention provides a method for administering a vector to a site within a patient using a device comprising an electromagnet and an element suitable for bringing the vector into proximity with the site, comprising the steps of:
adhering the vector to the element using the electromagnet;
inserting the element into the body of the patient; and
using the electromagnet to control the release of the vector from the element,
   wherein the vector comprises a binding moiety and a therapeutic agent and wherein the binding moiety comprises a metal-binding protein, polypeptide or peptide which is bound to or encapsulates a magnetic or magnetisable substance.

An alternative aspect of the present invention provides a method for removing a vector from a site within a patient using a device comprising an electromagnet and an element suitable for bringing the vector into proximity with the site, comprising the steps of:
inserting the element into the body of the patient;
adhering the vector to the element using the electromagnet; and
removing the element and the vector from the body of the patient,
   wherein the vector comprises a binding moiety and a therapeutic agent and wherein the binding moiety comprises a metal-binding protein, polypeptide or peptide which is bound to or encapsulates a magnetic or magnetisable substance.

### DETAILED DESCRIPTION OF THE INVENTION

The invention will now be described in further detail with reference, by way of example only, to the accompanying drawings.

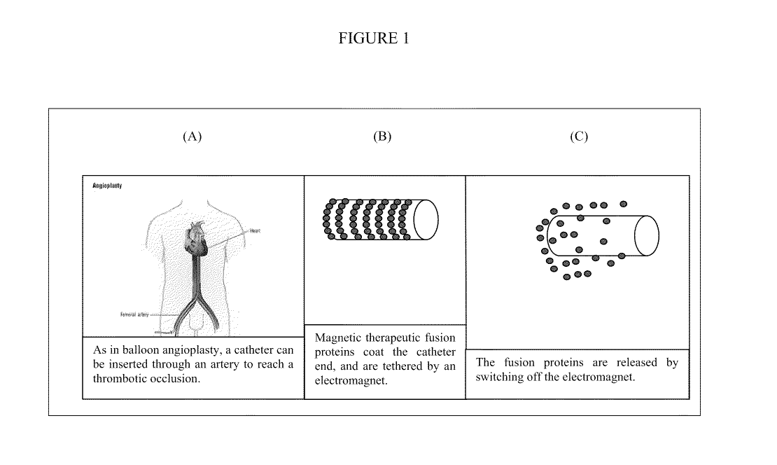

FIG. 1: this Figure shows a schematic of an embodiment of the present invention in which: (A) as in balloon angioplasty, a catheter is inserted through an artery to reach a thrombotic occlusion; (B) vectors according to the present invention comprising a fusion proteins encapsulating magnetic particles are tethered to the outer surface of the catheter using an electromagnet; and (C) when the catheter is in position the vectors are released by switching off the electromagnet.

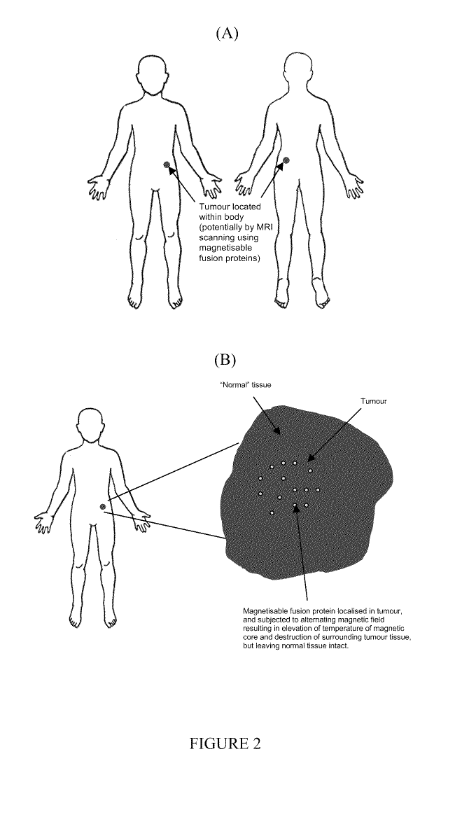

FIG. 2: this Figure shows a schematic of an embodiment of the present invention in which a tumour is treated to hyperthermia using the pharmaceutical composition of the present invention. In particular, this figure shows (A) the location of a tumour within a human body; (B) the localising of a pharmaceutical composition according to the present invention to the tumour and the treatment of the body with an alternating magnetic field; and (C) the destruction of the tumour by localised hyperthermia leaving the normal tissue intact.

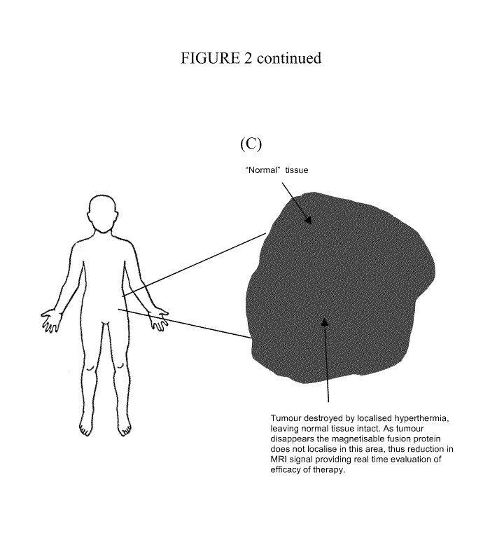

FIG. 3: this Figure shows how the appropriate genes are cloned into a plasmid or similar in order to produce the fusion protein of the binding moiety and the recognition moiety to be used in the vector for the present invention. The number of magnetisable protein units in the fusion protein may be controlled by including as many copies of the appropriate gene as necessary. Only genes for the V_{H} and V_{L} regions of the antibody are included in this example, so that the scFv portion of the antibody is included in the final preferred chimaeric protein, rather than the full antibody.

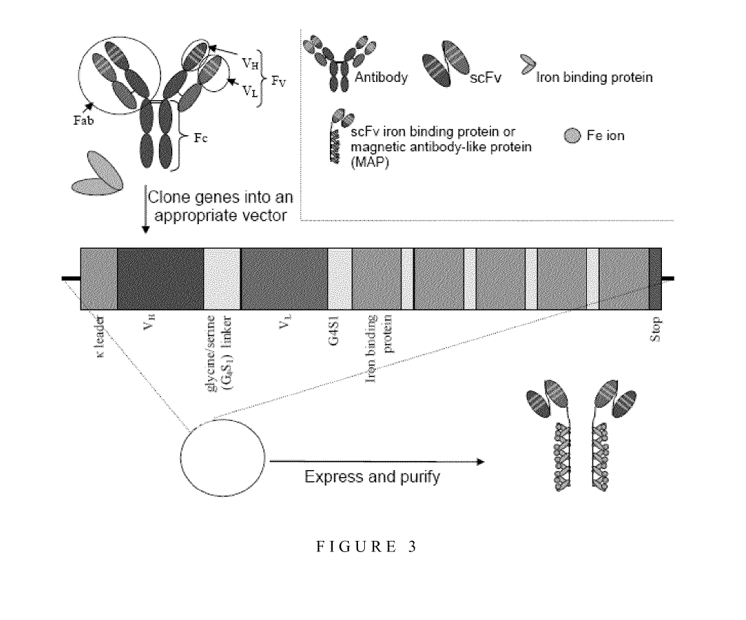

FIGS. 4a and 4b: these Figures schematically depict a simplification of the structure of antibodies such as IgG. After protease treatment using enzymes such as papain, antibodies are split into 3 parts close to the hinge region. As the effector function part of antibodies (the hinge, C_{H}2 and C_{H}3) are relatively easy to crystallise for X-ray diffraction analysis, this part has become known as the crystallisable fragment (Fc) region. The antigen binding portions of antibodies are known as the antibody fragment (Fab). After enzymic digestion, the Fab fragments can be linked at the hinge region thereby forming a F(ab)₂ fragment. Other antibodies may have differences in the number of domains in the Fc region and variations in the hinge region.

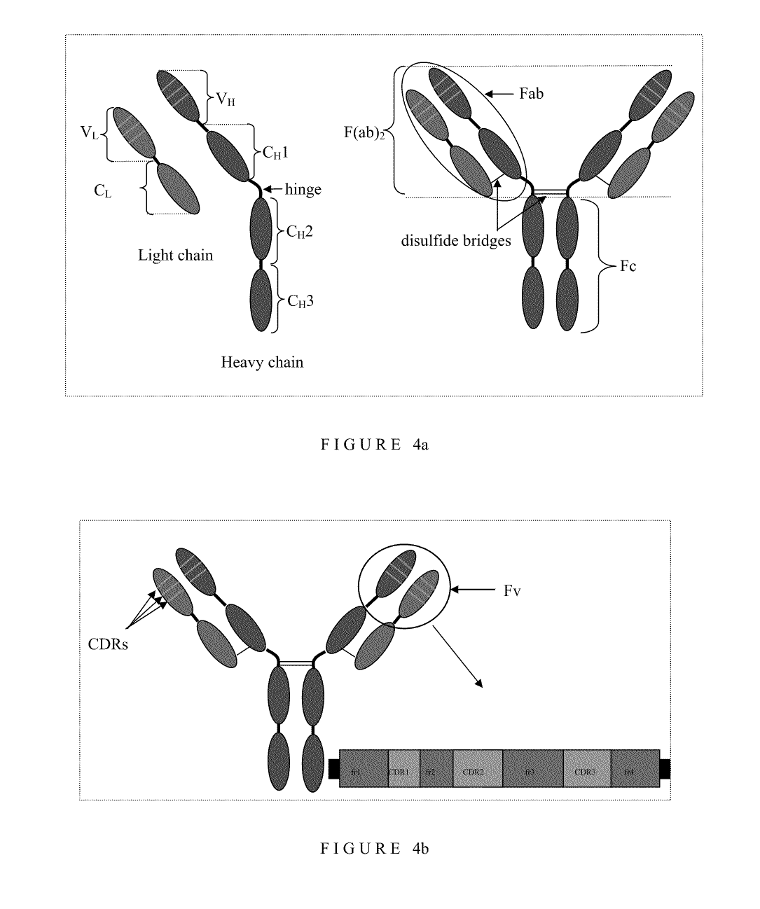

FIGS. 5a and 5b: these Figures show the construction of a scFv-ferritin fusion protein.

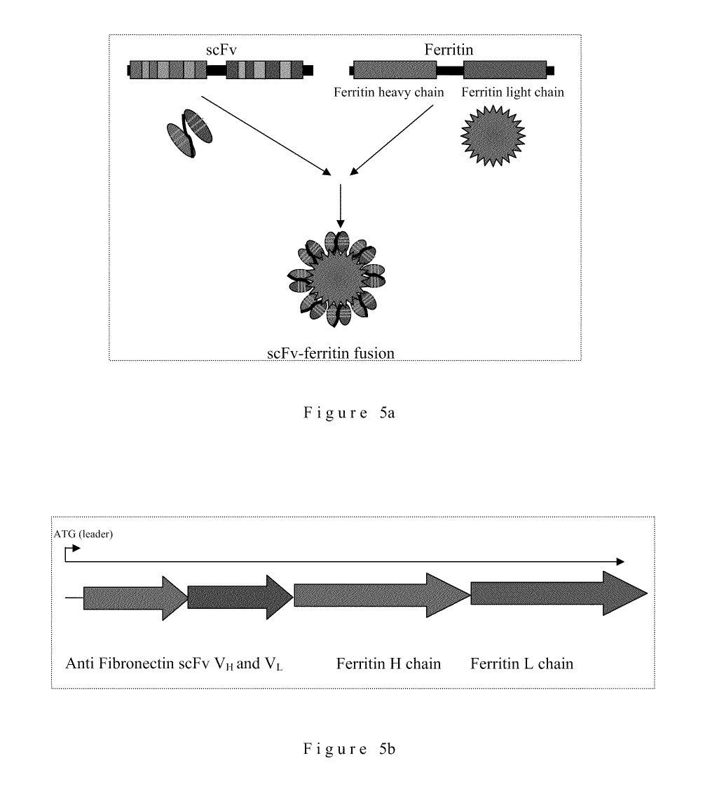

FIGS. 6a and 6b: these Figures show the construction of a scFv-MT2 fusion protein.

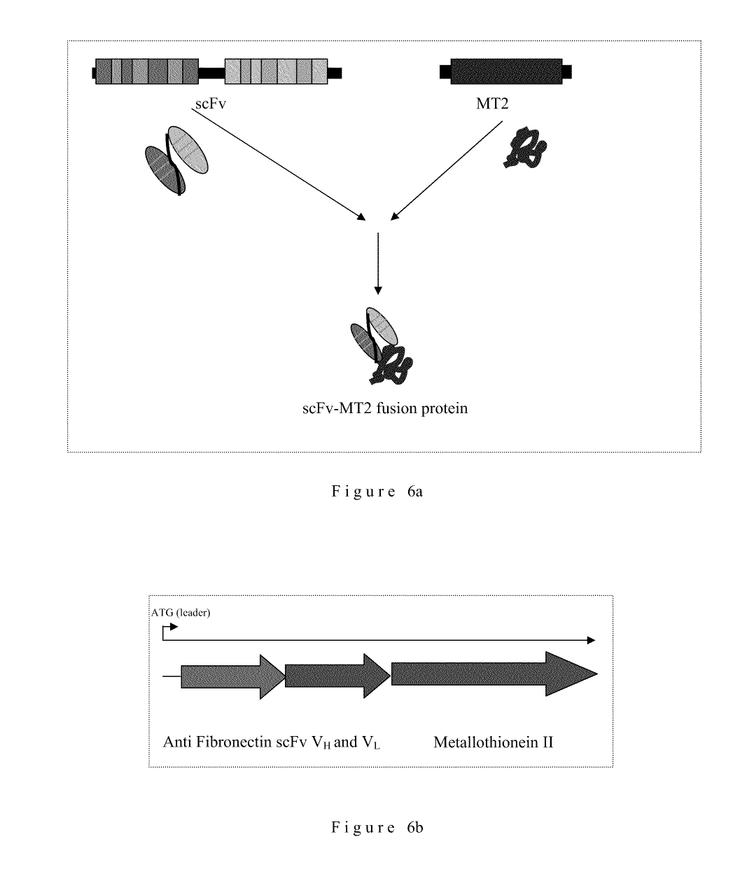

FIG. 7: this Figure shows the construction of a scFv fragment.

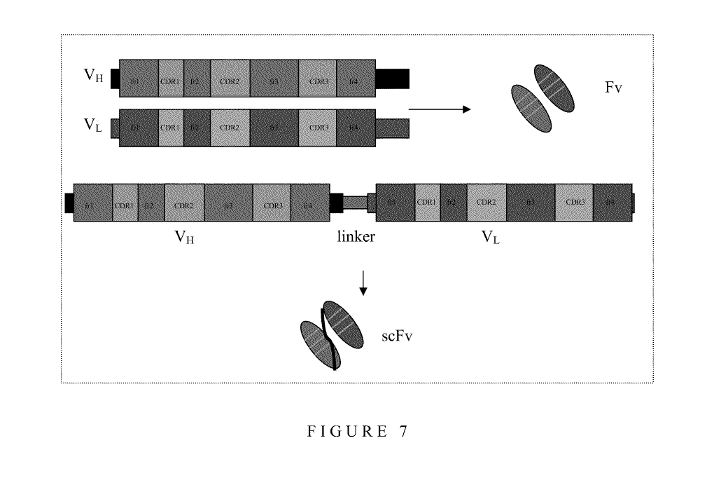

FIG. 8: this Figure shows construction of a cDNA library. In order to construct a cDNA library from a tissue sample, mRNA is extracted, reverse transcribed into cDNA and ligated into plasmid vectors. These vectors are then used to transform bacteria cells. The transformed cells are stored frozen until required. The frozen cells can be expanded by growing in appropriate media and the plasmids purified. Genes of interest can then be PCR amplified for further analysis using specific primer pairs.

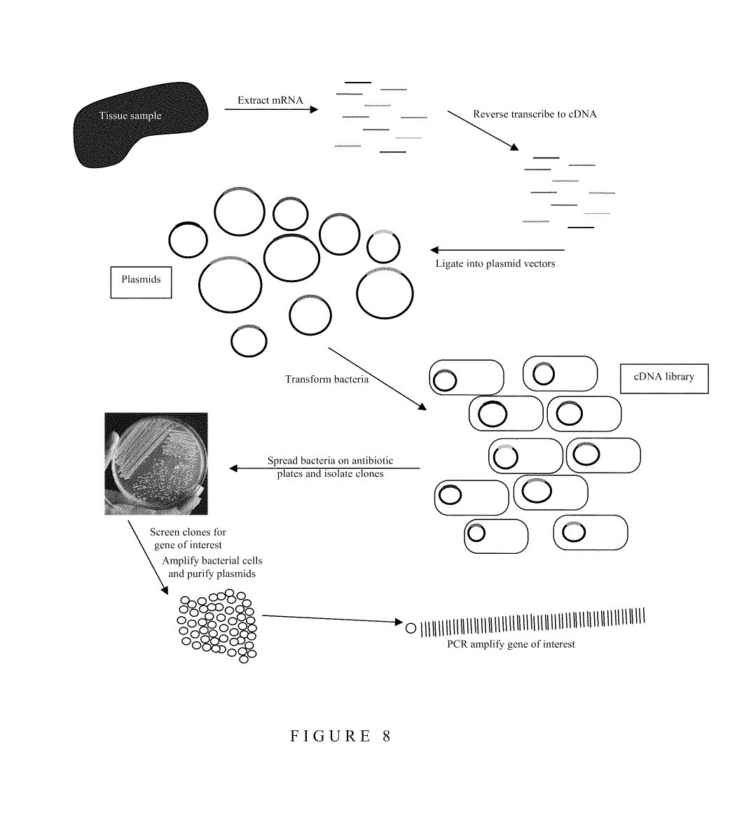

FIGS. 9a and 9b: these Figures show PCR amplicons of the ferritin heavy (H) and light (L) chain genes, and the overlapped PCR product of ferritin heavy and light chain genes, respectively. FIG. 9c shows colony PCR results, clones 1, 3 and 4 were selected for sequencing.

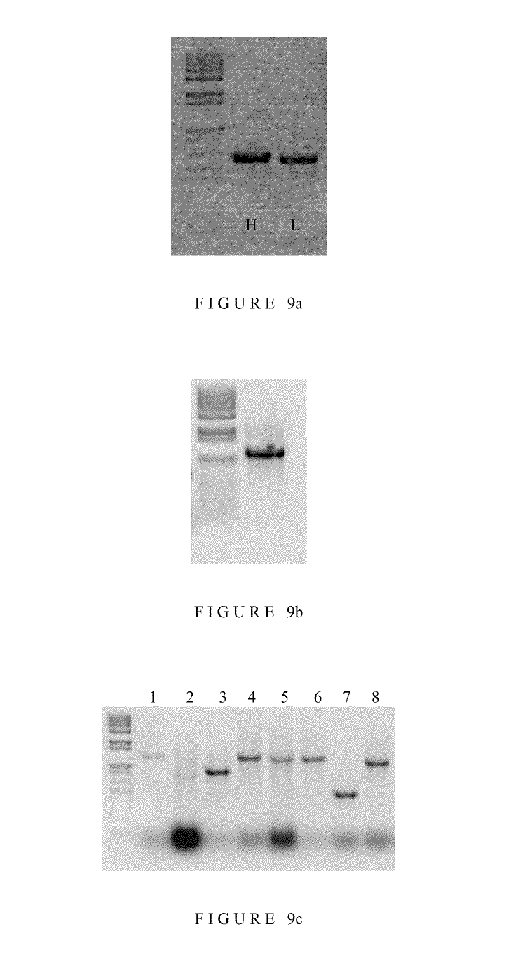

FIGS. 10a and 10b: these Figures show a gel showing the products of a PCR amplification of the anti-fibronectin scFv and ferritin heavy and light polygene (arrowed), and a gel showing the overlap PCR products, respectively.

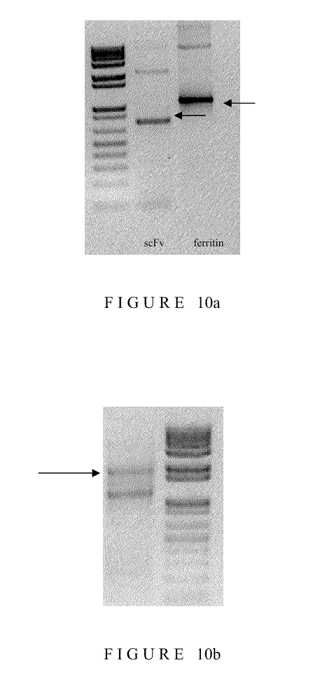

FIG. 11: this Figure shows a gel showing the results of a PCR screen of a number of clones transformed using plasmids that had been ligated with the scFv:ferritin fusion constructs.

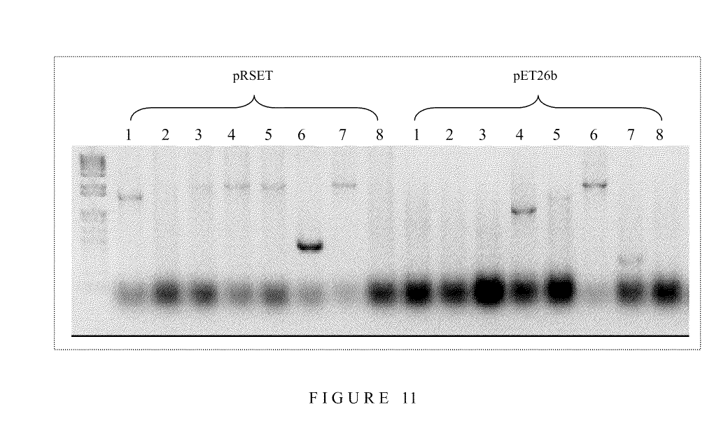

FIG. 12: this Figure shows Coomassie blue stained gel and Western blot of cell lysates respectively. Key: 1. Ferritin 2 hour induction; 2. Ferritin 3 hour induction; 3. Ferritin 4 hour induction; 4. Benchmark (Invitrogen) Protein Ladder.

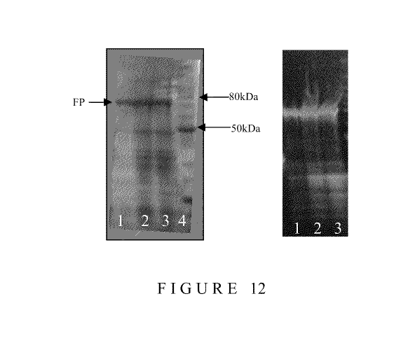

FIG. 13: this Figure shows a gel showing the PCR amplification product of MT2 from a human liver library.

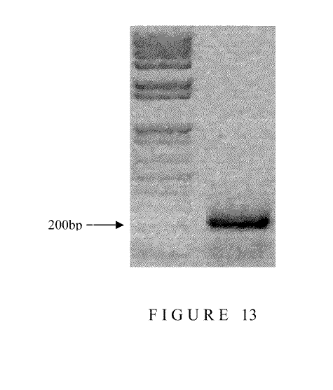

FIG. 14: this Figure shows colony analysis of clones transformed with plasmid containing the scFv:MT2 construct.

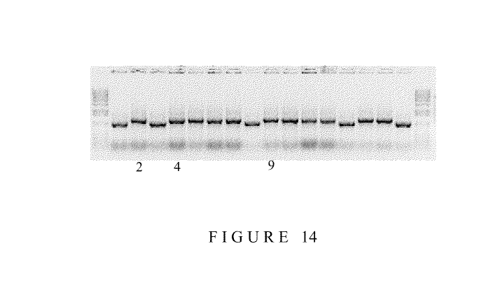

FIG. 15: this Figure shows (respectively) Coomassie gel and western blot of scFv:MT2 (arrowed).

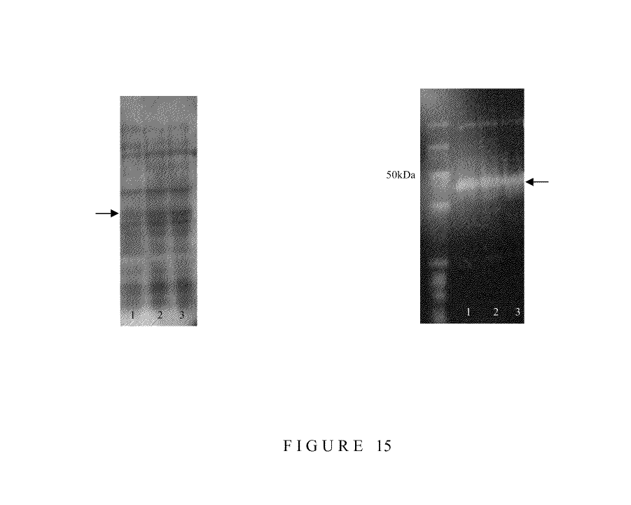

FIG. 16: this Figure shows photographs of a Coomassie blue stained gel and western blot (respectively) of the re-solubilised scFv:ferritin and scFv:MT2 fusion proteins. The fusion proteins are circled—ferritin is in lane 2 on both gels and MT2 is in lane 3 of both gels. A protein molecular weight ladder is in lane 1.

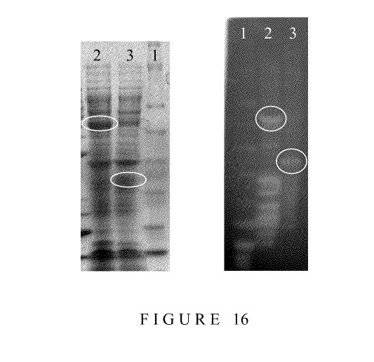

FIGS. 17a and 17b: these Figures show overlaid Sensograms from the SPR analysis of the binding of MT2 and ferritin fusion proteins respectively.

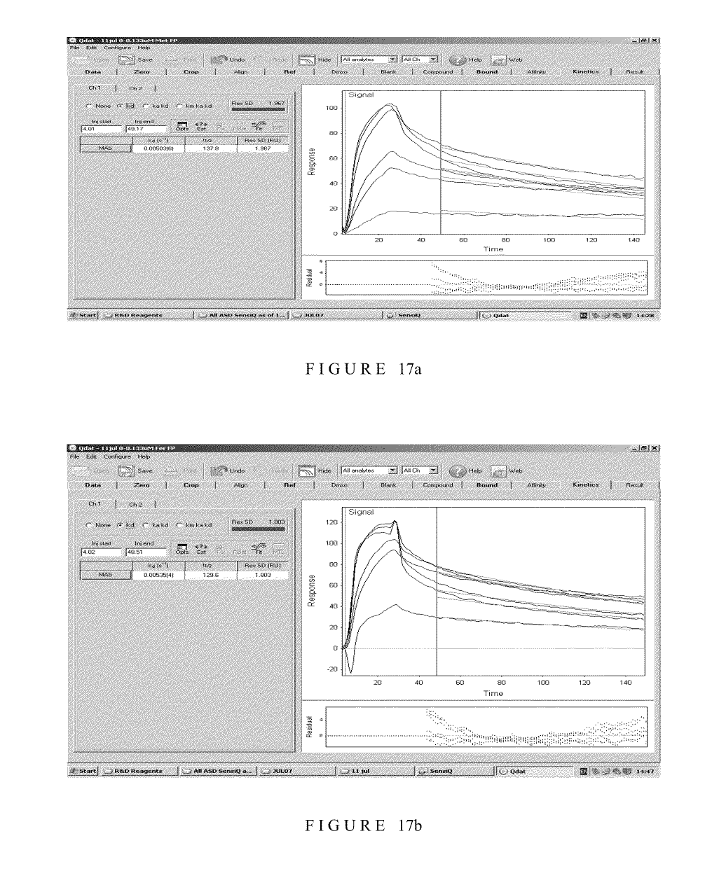

FIG. 18: this Figure demonstrates the magnetic nature of the magnetoferritin produced for use in the present invention.

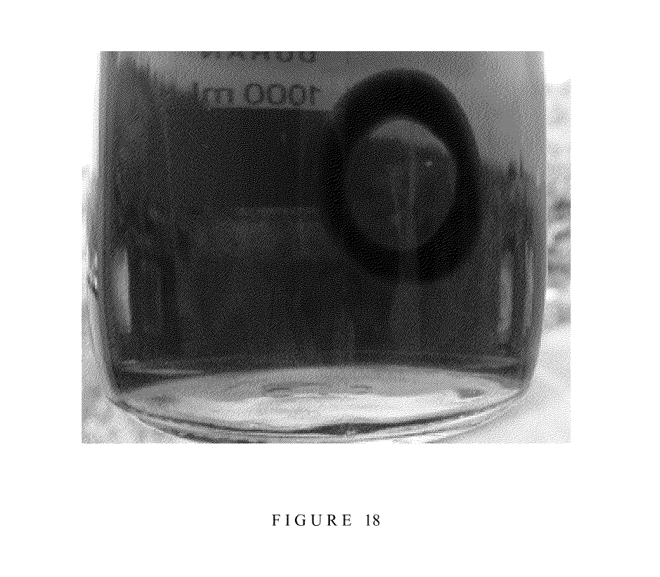

FIG. 19: this Figure shows the concentration of ferritin during the production and concentration of magnetoferritin. Key: MF; Magnetoferritin: ft; Flow-through: Pre; pre-dialysis Macs® column concentrated magnetoferritin: post; post-dialysis Macs® column concentrated magnetoferritin.

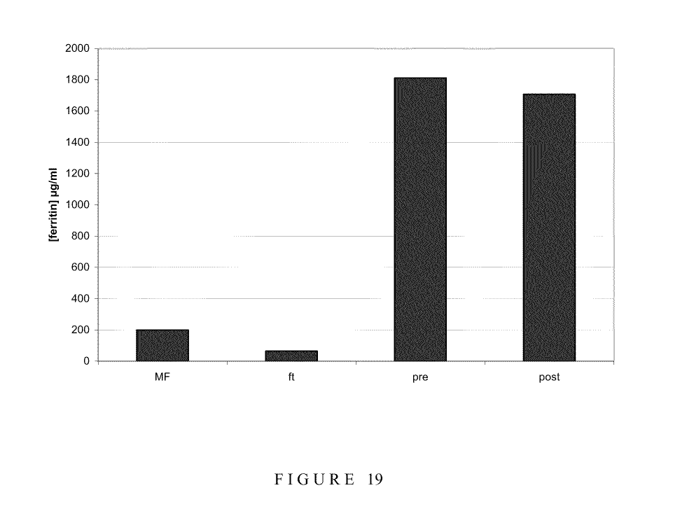

FIG. 20: this Figure shows binding of scFv:ferritin and heat treated scFv:ferritin to fibronectin.

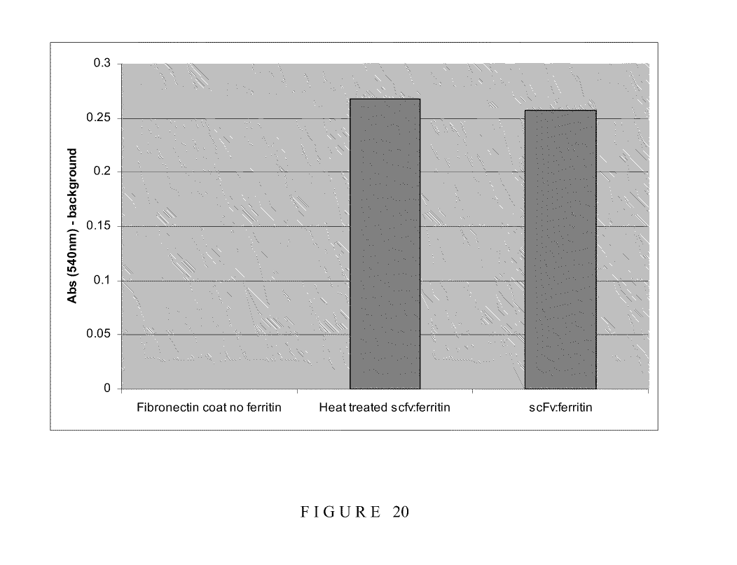

FIGS. 21a and 21b: these Figures show absorbance measurements, recorded using a Varioskan Flash instrument, on magnetised fusion protein. After concentration the protein is still recognised by the monoclonal anti-ferritin antibody (21a) and the magnetised anti-fibronectin ferritin fusion protein retains binding ability to its target antigen (21b).

As described above, the present invention relates to a vector for use in delivering a therapy to or removing a therapy from a site within a patient, wherein the vector comprises:
a) a binding moiety; and
b) a therapeutic agent,
   wherein the binding moiety comprises a metal-binding protein, polypeptide or peptide which is bound to or encapsulates a magnetic or magnetisable substance,
   and wherein the vector is to be administered using a device comprising an electromagnet and an element suitable for bringing the therapeutic agent into proximity with the site.

The vector is to be delivered utilising a device comprising an electromagnet and an element suitable for bringing the therapeutic agent into proximity with the site. In a preferred embodiment the element is to be inserted into the body of the patient. In particular, the element to be is preferably a catheter (for example the type of catheter routinely used in angioplasty/brachytherapy procedures) with an electromagnet at one end to physically deliver the vector to an area to be treated. As shown in FIG. 1, once the electromagnet is turned off, the magnetic vector carrying the therapeutic agent can pass into the surrounding tissue.

The binding moiety which binds to or encapsulates the magnetic or magnetisable substance is not especially limited, provided that it is non-toxic, capable of binding the substance and can be attached to the therapeutic agent. The binding moiety comprises a metal-binding protein, polypeptide or peptide (or the metal-binding domain of such a protein polypeptide or peptide). The binding moiety should be capable of binding or encapsulating (or otherwise attaching in a specific or non-specific manner) to the magnetic or magnetisable substance in the form of particles or aggregates or the like.

These particles or aggregates typically have less than 100,000 atoms, ions or molecules, more preferably less than 10,000 atoms, ions or molecules, and most preferably less than 5,000 atoms, ions or molecules bound or encapsulated to the (or each) moiety in total. The most preferred substances are capable of binding up to 3,000 atoms, ions or molecules, and in particular approximately 2,000 or less, or 500 or less such species.

In one specific example employed in the invention, the metallic component of ferritin (a 24 subunit protein shell) consists of an 8 nm (8×10⁻⁹ m) inorganic core. Each core contains approximately 2,000 Fe atoms. In another example, Dpr, from Streptococcus mutans (a 12 subunit shell), consists of a 9 nm shell containing 480 Fe atoms. In a further example, lactoferrin binds 2 Fe atoms and contains iron bound to haem (as opposed to ferritin which binds iron molecules within its core). Metallothionein-2 (MT) binds 7 divalent transition metals. The zinc ions within MT are replaced with Mn²⁺ and Cd²⁺ to create a room temperature magnetic protein. MT may be modified to further incorporate one or more additional metal binding sites, which increases the magnetism of the Mn, Cd MT protein.

In accordance with these binding environments, the total volume of the substance bound or encapsulated in a single moiety typically does not exceed 1×10⁵ nm³ (representing a particle or aggregate of the substance having an average of about 58 nm or less). More preferably the substance may have a total volume of not more than 1×10⁴ nm³ (representing a particle or aggregate of the substance having an average diameter of about 27 nm or less). More preferably still the substance may have a total volume of not more than 1×10³ nm³ (representing a particle or aggregate of the substance having an average diameter of about 13 nm or less). Most preferably the substance may have a total volume of not more than 100 nm³ (representing a particle or aggregate of the substance having an average diameter of 6 nm or less). However, the size of the particles may be determined by average diameter as an alternative to volume. It is thus also preferred in the present invention that the average diameter of the bound particles is 50 nm or less, 40 nm or less, 30 nm or less, 20 nm or less or most preferably 10 nm or less. In this context, average means the sum of the diameters of the number of particles, divided by the number of particles.

In a particularly preferred embodiment of the invention the magnetic or magnetisable substance is paramagnetic and only exhibits magnetism under the influence of a stronger magnet.

Typically the binding moiety is bound to or encapsulates one or more transition and/or lanthanide metal atoms and/or ions, or any compound comprising such ions. Such ions include, but are not limited to, any one or more ions of Fe, Co, Ni, Mn, Cr, Cu, Zn, Cd, Y, Gd, Dy, or Eu.

In the more preferred embodiments of the invention, the one or more metal ions comprise any one or more of Fe²⁺, Fe³⁺, Co²⁺, Co³⁺, Mn²⁺, Mn³⁺, Mn⁴⁺, Cd²⁺ and Ni²⁺. The most preferred ions for use in the present invention are Fe²⁺ and Fe³⁺ and Cd²⁺ and Mn²⁺ ions. Typically these ions are bound by lactoferrin, transferrin and ferritin in the case of iron, and metallothionein-2 in the case of cadmium and manganese. The binding of Fe²⁺ is preferably promoted by employing acidic conditions, whilst the binding of Fe³⁺ is preferably promoted by employing neutral or alkaline conditions.

In preferred embodiments of the invention, the metal-binding moiety comprises a protein, or a metal-binding domain of a protein, selected from lactoferrin, transferrin, ferritin (apoferritin), a metallothionein (MT1 or MT2), a ferric ion binding protein (FBP e.g. from Haemophilus influenzae), frataxin and siderophores (very small peptides which function to transport iron across bacterial membranes).

In a particularly preferred embodiment of the metal-binding moiety comprises ferritin or metallothionein II (MT2), or fragments or segments thereof. Ferritin is a large protein, 12-nm diameter, with a molecular weight of 480 kDa. The protein consists of a large cavity (8 nm diameter) which is able to store thousands of iron ions. As the endogenous iron within ferritin is not paramagnetic, it typically needs to be removed and replaced with a paramagnetic form without damaging the protein. Ferritin is a large multifunctional protein with eight Fe transport pores, 12 mineral nucleation sites and up to 24 oxidase sites that produce mineral precursors from ferrous iron and oxygen. Two types of subunits (heavy chain (H) and light chain (L)) form ferritin in vertebrates, each with catalytically active (H) or inactive (L) oxidase sites. The ratio of heavy and light chains varies according to requirements. Up to 4000 iron atoms can be localised in the centre of the ferritin protein.

The iron stored within ferritin is usually in the form of hydrated iron oxide ferrihydrite (5Fe₂O₃.9H₂O). It is possible to replace the ferrihydrite core with ferrimagnetic iron oxide, magnetite (Fe₃O₄). This may be achieved by removing the iron using thioglycolic acid to produce apoferritin. Fe(II) solution is then gradually added under argon or other inert gas with slow, controlled oxidation by the introduction of air, or an alternative oxidising agent.

In contrast the protein metallothionein II holds fewer ions of metal in a loose lattice arrangement, making it potentially easier to remove and replace these than with ferritin. Metallothioneins are intracellular, low molecular weight, cysteine-rich proteins.

MT2 binds seven divalent transition metals via two metal binding clusters at the carboxyl α-domain) and amino (β-domain) terminals. Twenty cysteine residues are involved in the binding process.

Chang et al describe a method of replacing the seven zinc (Zn²⁺) ions with manganese (Mn²⁺) and cadmium (Cd²⁺) ions. The resultant protein was shown to exhibit a magnetic hysteresis loop at room temperature. This could potentially mean that the protein is paramagnetic.

Toyama et al engineered human MT2 to construct an additional metal binding site. This could potentially increase the paramagnetic functioning of the MT2, and may be employed in the present invention.

In some embodiments, the vector of the invention may comprise a plurality of binding moieties binding or encapsulating the magnetic or magnetisable substance. The number of such moieties may be controlled so as to control the magnetic properties of the vector.

Typically in such embodiments, the vectors may comprise from 2-100 such moieties, preferably from 2-50 such moieties and most preferably from 2-20 such moieties for binding the magnetic or magnetisable substance. In the final chimaeric protein, each copy of the metal-binding protein may be attached to the next by non-charged amino acid linker sequences for flexibility.

In further embodiments modifications such as glycosylation or phosphorylation may be made to the protein/peptide binding moieties so as to adjust their (electro)magnetic properties.

The therapeutic agent carried by the vector is not particularly limited provided it is capable of being attached to the binding moiety. By ‘attached to’ in the present context, it is meant that the attachment is of any type, including specific and non-specific binding and also encapsulation. Thus, the binding moiety should be capable of binding or encapsulating (or otherwise attaching in a specific or non-specific manner) the therapeutic agent to enable the vector to transport or carry the agent. In a particular aspect of the invention the therapeutic agent is formed as a fusion protein with the binding moiety.

In a specific embodiment of the present invention the magnetic properties of the magnetic or magnetisable substance are utilised in the therapy, and the magnetic or magnetisable substance is the therapeutic agent. In particular, as will be described in more detail below, the vector comprising the magnetic or magnetisable substance localised at a particular site within a body can be used in conjunction with the treatment of the body with a magnetic field. The field causes heating of the magnetic or magnetisable substance resulting in hyperthermia at the site. Such hyperthermia can be used to destroy cells at the site. In this embodiment the magnetic or magnetisable substance is suitable for delivering hyperthermic therapy to the site on exposure to an electromagnetic field.

In another embodiment of the present invention the therapeutic agent is selected from a radio-isotope, a chemotherapeutic agent, a thrombolytic or anti-thrombotic agent, and an anti-angiogenic agent. In a preferred embodiment the therapeutic agent is antithrombin III and the therapy is the treatment of blood clots. Utilising the present invention to deliver anti-clotting agents directly to the site of blood clots is advantageous over systemically delivered anti-clotting agents. In particular, the present invention allows the use of a lower dose of anti-clotting agent (while still maintaining the localised therapeutic concentration) and reduces the risk associated with anti-clotting treatment; for example, the risk of excessive bleeding in non-target tissues (particularly the brain).

In a preferred embodiment of the present invention the vector also comprises one or more recognition moieties which are capable of binding to one or more targets present on a diseased cell or in a diseased tissue. The presence of at least one recognition moiety as part of the vector can provide additional targeting of the vector. Examples of possible targets may be an infectious agent or component of an infectious agent (such as a virus or virus particle or virus component), a cellular component present on the surface of a cell, a small molecule such as an endogenous or exogenous small molecule (e.g. a metabolite, or a pharmaceutical or drug), or a body such as a blood clot. Intracellular targeting is also possible. A nuclear localisation signal can be used as recognition moiety to target the vector to the nucleus. Alternatively, recognition moieties can be selected to target the vector to the Golgi apparatus or the inner cell membrane. Targeting to the inner cell membrane can be particularly effective where it is desired that the vector release the therapeutic agent, and that this diffuses out of the cell into the surrounding tissue.

In particular, the recognition moiety may recognise an antigen expressed on the surface of a tumour cell. Some tumours express a variety of antigens on their surface. Accordingly, it is particularly preferred that the vector comprises at least two recognition moieties which recognise and bind at least two different antigens on the surface of a tumour cell. In an alternative arrangement the vector comprises at least two recognition moieties to achieve receptor cross-linking. For example, one of the recognition moieties can recognise a tumour marker while the other recognises, binds and activates Fc receptors on an immune cell. The immune cell then “recognises” the tumour cell and will kill it. Such receptor cross-linking is advantageous since it increases the selectivity of treatment.

The recognition moiety that is capable of binding to the targets described above may itself be any type of substance or molecule, provided that it is suitable for binding to the target. Generally, the recognition moiety is selected from an antibody or a fragment of an antibody, a receptor or a fragment of a receptor, a protein, a polypeptide, a peptidomimetic, a nucleic acid, an oligonucleotide and an aptamer. More specifically the recognition moiety is selected from an antibody or a fragment of an antibody, a receptor or a fragment of a receptor, a protein, and a polypeptide. In more preferred embodiments of the invention, the recognition moiety is selected from a variable polypeptide chain of an antibody (Fv), a T-cell receptor or a fragment of a T-cell receptor, avidin, streptavidin and heparin. Most preferably, the recognition moiety is selected from a single chain of a variable portion of an antibody (sc-Fv).

In a particularly preferred aspect of the present invention the binding moiety and the recognition moiety together form a fusion protein. In the context of the present invention, a fusion protein is a protein that has been expressed as a single entity recombinant protein. The fusion protein may be generated from any known expression system. However, in a preferred aspect of the present invention the fusion protein of the vector is produced in a mammalian expression system. It is preferred that the binding moiety and the recognition moiety in the fusion protein are separated by a linker. The linker is typically less than 15 amino acid, preferably less than 10 amino acids and most preferably less than 5 amino acids in length.

In an embodiment of this preferred aspect the vector comprises a binding moiety which is a plurality of ferritin subunits, which assemble to form a particle, with the recognition moieties present on the outer surface thereof. Such a particle may encapsulate magnetic or magnetisable material together with an additional therapeutic agent.

The use of fusion proteins in the vector creates a number of further advantages. The orientation of the recognition arm of the fusion protein (e.g. the scFv) within the vector will be controlled and therefore more likely to bind its target. Fusion proteins also facilitate the possibility of incorporating a plurality of recognition moieties in a single fusion protein. These recognition sites may be directed against the same target or to different targets.

The use of fusion proteins creates particular advantages where the binding moiety is made up of several subunits which assemble together to form a particle, as is the case where the binding moiety comprises ferritin. During production, genetically engineered nucleotide sequences, which encode the binding moiety, are expressed in vitro. The proteins/peptides produced are subjected to conditions which enable them to assemble into particles. Different nucleotide sequences can be expressed together so as to create a multifunctional particle. For example, a sequence encoding ferritin alone can be expressed with a sequence encoding a fusion protein of ferritin and a recognition moiety, so as to create a particle on which only some of the subunits display the recognition moiety. The ratios of the different nucleotide sequences to be expressed can be controlled so as to obtain assembled particles displaying an optimum number of recognition moieties. Such a system can be used to minimise the effect of steric hinderance and optimise the binding of the particle to the target.

As indicated above, in an preferred aspect the vector comprises one or more recognition moieties, which are preferably antibodies or antibody fragments. Antibodies are immunoglobulin molecules involved in the recognition of foreign antigens and expressed by vertebrates. Antibodies are produced by a specialised cell type known as a B-lymphocyte or a B-cell. An individual B-cell produces only one kind of antibody, which targets a single epitope. When a B-cell encounters an antigen it recognises, it divides and differentiates into an antibody producing cell (or plasma cell).

The basic structure of most antibodies is composed of four polypeptide chains of two distinct types (FIG. 4). The smaller (light) chain being of molecular mass 25 kilo-Daltons (kDa) and a larger (heavy) chain of molecular mass 50-70 kDa. The light chains have one variable (V_{L}) and one constant (C_{L}) region. The heavy chains have one variable (V_{H}) and between 3-4 constant (C_{H}) regions depending on the class of antibody. The first and second constant regions on the heavy chain are separated by a hinge region of variable length. Two heavy chains are linked together at the hinge region via disulfide bridges. The heavy chain regions after the hinge are also known as the Fc region (crystallisable fragment). The light chain and heavy chain complex before the hinge is known as the Fab (antibody fragment) region, with the two antibody binding sites together known as the F(ab)₂ region. The constant regions of the heavy chain are able to bind other components of the immune system including molecules of the complement cascade and antibody receptors on cell surfaces. The heavy and light chains of antibodies form a complex often linked by a disulfide bridge, which at the variable end is able to bind a given epitope (FIG. 4).

The variable genes of antibodies are formed by mutation, somatic recombination (also known as gene shuffling), gene conversion and nucleotide addition events.

ScFv antibodies may be generated against a vast number of targets including:
1. Viruses: Torrance et al. 2006. Oriented immobilisation of engineered single-chain antibodies to develop biosensors for virus detection. J Virol Methods. 134 (1-2) 164-70.
2. Hepatitis C virus: Gal-Tanamy et al. 2005. HCV NS3 serine protease-neutralizing single-chain antibodies isolated by a novel genetic screen. J Mol Biol. 347 (5):991-1003), and Li and Allain. 2005 Chimeric monoclonal antibodies to hypervariable region 1 of hepatitis C virus. J Gen Virol. 86 (6) 1709-16.
3. Cancers: Holliger and Hudson. Engineered antibody fragments and the rise of single domains. Nat Biotechnol. 23 (9) 1126-36.

Thus, in its most preferred embodiments, the present invention makes use of a multi-recognition moiety vector typically formed from one or more antigen binding arms of one or more antibodies, for recognising one or more targets within the body, and one or more copies of a metal-binding protein attached to the antigen binding arm. Typically the antibody fragment used comprises the variable regions of the heavy and light chains, V_{H} and V_{L} joined by a flexible linker to create a single chained peptide (sc), usually termed scFv. When both the recognition and binding moieties in the vector are formed from protein and/or polypeptides (i.e. the vector comprises a chimaeric protein) the vector may be partially formed using recombinant techniques that are well known in the art. An illustration of this is provided in FIG. 3. However, should any of the recognition moieties be formed from other species, the vectors may be made by simple attachment of one species to the binding moiety.

The vectors of the present invention may optionally incorporate specific cleavage sites between the binding moiety and the recognition moiety, within the recognition moiety or, where the binding moiety is an assembled particle, between subunits of the particle, so as to allow the vector to be broken down if required. This can particularly be achieved by incorporating specific protease cleavage sites into the vector.

For example, the subunits of the binding moiety can be linked by a length of amino acid residues which provide a cleavage site for a specific protease. During use, when the vector is exposed to the protease, it will be broken down, thus releasing the encapsulated therapeutic agent. Specific cleavage sites can be used which are only recognised in particular cell types or tissues, leading to selective release of the therapeutic agent. Alternatively, the cleavage site may be within the recognition moiety such that action by a protease can remove the upper segment of the recognition moiety to “reveal” a second recognition moiety with different specificity.

A further aspect of the invention provides pharmaceutical compositions that are of particular use in the invention described above. In particular, a pharmaceutical composition is provided comprising:
(a) a binding moiety;
(b) a therapeutic agent; and
(c) a recognition moiety
   wherein the binding moiety comprises a metal-binding protein, polypeptide or peptide which is bound to or encapsulates a magnetic or magnetisable substance.

It is particularly preferred that the binding moiety and the recognition moiety form parts of a fusion protein of the type described above.

The descriptions of the binding moiety, therapeutic agent, recognition moiety and the magnetic or magnetisable substance of the vector provided above also apply to the pharmaceutical composition.

The pharmaceutical compositions of the present invention may also comprise a further component selected from an excipient, a carrier, a solvent, a diluent, an adjuvant and a buffer.

In one embodiment the magnetic or magnetisable substance is utilised as the therapeutic agent, while in an alternative embodiment the pharmaceutical composition comprises a therapeutic agent attached to the binding moiety which is not the magnetic or magnetisable substance.

Where the magnetic or magnetisable substance is the therapeutic agent the pharmaceutical composition is for use in treating diseased cells or tissue susceptible to hyperthermia in a patient, wherein the composition is to be administered prior to exposing the patient to an electromagnetic field. In this embodiment the magnetic or magnetisable substance is suitable for delivering hyperthermic therapy to the patient on exposure to an electromagnetic field.

It has been suggested in the art that ferritin-mediated electromagnetic hyperthermia can be used in the selective treatment of neoplastic cells (Babincová M. et al., Medical Hypotheses (2000) Volume 54, No. 2, pages 177-179). In particular, it is know that cancer cells are more sensitive to high temperatures than normal non-cancerous cells. Babincová et al., described earlier studies in which a tumour body is injected with a magnetic fluid comprising suspensions of ferromagnetic or ferrite particles. The particles are heated up by applying an alternative magnetic field. Babincová et al., propose a gene therapy technique in which the ferritin gene is delivered specifically to cancer cells within a patient to increase the level of ferritin in these malignant cells.

The present invention provides an alternative approach using the pharmaceutical composition described above. In particular, the recognition moiety can be utilised to deliver the magnetic or magnetisable substance, bound to or encapsulated by the binding moiety, to the cancer cells, as shown in FIG. 2. Specifically, the recognition moiety is targeted against markers expressed on cancerous cells, such that the magnetic or magnetisable substance is concentrated in the tumour. The application of an alternating magnetic field results in heating of the magnetic or magnetisable substance within the binding moiety, and thus elevation of temperature. This leads to death of the neoplastic cells whilst non-neoplastic cells are unaffected and remain viable.

In alternative embodiments of this aspect of the invention the pharmaceutical compositions can also be used to treat other types of abnormal tissue growth or function within the body. For example, the compositions can be used to treat conditions such as hyperthyroidism, cyst growth and atherosclerotic plaques. In particular, compositions comprising a recognition moiety which targets thyroid tissue may be used to destroy or remove part of the thyroid gland in the treatment of hyperthyroidism. Further, atherosclerotic plaques are associated with an increase in a number of localised markers. A composition comprising a recognition moiety which targets one or more of these markers could be used to disrupt the plaques (particularly at an early stage in their formation).

While the pharmaceutical agents in the above paragraphs are localised using a recognition moiety they may preferably also be targeted using the method of the present invention involving an electromagnet.

The invention will now be described in more detail, by way of example only, with reference to the following example.

### EXAMPLES

### Example 1

### Treatment of a Blood Clot

It is envisaged that the vector of the present invention will be used to treat a blood clot within a blood vessel in a patient. The vector will comprise a fusion protein comprising ferritin and an antithrombin III antibody fragment. The vector will further comprise Fe₂O₃ as the magnetic or magnetisable substance.

The vector will be attached to an electromagnet on the outside of one end of a catheter and the catheter will be inserted into the patient through the femoral artery. Once the catheter has been positioned as close to the site of the blood clot as is possible, the electromagnet will be switched off, releasing the vector. It is envisaged that the antithrombin III part of the fusion protein will bind to the enzymes of the coagulation system preventing further clotting.

The following experimental detail indicates how the fusion protein of a recognition moiety and a binding moiety can be made:

### Example 2

### Design and Manufacture of Fusion Proteins

In order to exemplify the invention, fusion proteins were designed, using commercially available murine anti-fibronectin antibody. Fusion proteins consisting of anti-fibronectin scFv genetically linked by short flexible linkers to either MT2, or ferritin were produced. This Example details the construction of the fusion proteins, their characterisation and isolation.

The design of the anti-fibronectin ferritin or MT2 fusion proteins was based on cloning the V_{H} and V_{L} genes from a mouse anti-fibronectin antibody into a vector. Both genes were linked by short, flexible linkers composed of small non-charged amino acids. Immediately at the 3′ end of the V_{L} gene, another short flexible linker led into either the ferritin genes or the MT2 gene. Both fusion proteins had a six-histidine region for purification on nickel columns. The fusion protein translation was terminated at a stop codon inserted at the 3′ end of the ferritin light gene or the MT2 gene. The plasmid vector containing all these elements was used to transform bacteria for expression.

The genes for the ferritin and MT2 were obtained from cDNA libraries. A cDNA library is formed by obtaining mRNA from cells or tissues, reverse transcribing the RNA to cDNA using an enzyme known as reverse transcriptase and cloning each individual cDNA into a plasmid vector (see FIG. 8).

### Generation of the Anti-Fibronectin: Ferritin Fusion Protein

### Background

Ferritin is a 12-nm diameter protein with a molecular weight of approximately 480 kDa. The protein consists of a large cavity (8 nm diameter) which encases iron. The cavity is formed by the spontaneous assembly of 24 ferritin polypeptides folded into four-helix bundles held by non-covalent bonds. The amino acid sequence and therefore the secondary and tertiary structures of ferritin are conserved between animals and plants. The structure of the protein in bacteria is the same as eukaryotes, although the sequence is different. Two types of subunits (heavy chain (H) and light chain (L)) form ferritin in vertebrates, each with catalytically active (H) or inactive (L) oxidase sites. The ratio of heavy and light chains varies according to requirement. The amino acid sequences of the ferritin heavy and light chains used in the construction of the fusion proteins are:

Ferritin heavy chain (molecular weight 21096.5 Da):

(SEQ ID No: 3)
MTTASTSQVRQNYHQDSEAAINRQINLELYASYVYLSMSYYFDRDD
VALKNFAKYFLHQSHEEREHAKLMKLQNQRGGRIFLQDIKKPDCDD
WESGLNAMECALHLEKNVNQSLLELHKLATDKNDPHLCDFIETHYL
NEQVKAIKELGDHVTNLRKMGAPESGLAEYLFDKHTLGDSDNES

Ferritin light chain (molecular weight 20019.6 Da):

(SEQ ID No: 4)
MSSQIRQNYSTDVEAAVNSLVNLYLQASYTYLSLGFYFDRDDVALE
GVSHFFRELAEEKREGYERLLKMQNQRGGRALFQDIKKPAEDEWGK
TPDAMKAAMALEKKLNQALLDLHALGSARTDPHLCDFLETHFLDEE
VKLIKKMGDHLTNLHRLGGPEAGLGEYLFERLTLKHD

Together with the anti-fibronectin scFv amino acid sequences, the predicted sequence of a single polypeptide of the fusion protein is (with the linker sequences between the heavy and light antibody genes and between the antibody light chain and ferritin heavy chain highlighted in lower case):

(SEQ ID No: 1)
LVQPGGSLRLSCAASGFTFSSFSMSWVRQAPGKGLEWVSSISGSSGT
TYYADSVKGRFTSRDNSKNTLYLQMNSLRAEDTAVYYCAKPFPYFDY
WGQGTLVTVSSGDgssggsggASTGEIVLTQSPGTLSLSPGERATLS
CRASQSVSSSFLAWYQQKPGQAPRLLIYYASSRATGIPDRFSGSGSG
TDFTLTISRLEPEDFAVYYCQQTGRIPPTFGQGTKVEIKsgggMTTA
STSQVRQNYHQDSEAAINRQINLELYASYVYLSMSYYFDRDDVALKN
FAKYFLHQSHEEREHAKLMKLQNQRGGRIFLQDIKKPDCDDWESGLN
AMECALHLEKNVNQSLLELHKLATDKNDPHLCDFIETHYLNEQVKAI
KELGDHVTNLRKMGAPESGLAEYLFDKHTLGDSDNESMSSQIRQNYS
TDVEAAVNSLVNLYLQASYTYLSLGFYFDRDDVALEGVSHFFRELAE
EKREGYERLLKMQNQRGGRALFQDIKKPAEDEWGKTPDAMKAAMALE
KKLNQALLDLHALGSARTDPHLCDFLETHFLDEEVKLIKKMGDHLTN
LHRLGGPEAGLGEYLFERLTLKHD

The molecular weight of the polypeptide component was 65.550 kDa.

### Assembly of the Anti-Fibronectin: Ferritin Fusion Protein Genes

Ferritin heavy and light chain genes were amplified from a human liver cDNA library using PCR (see FIG. 9a). The PCR products were of the expected size (˜540 bp). These PCR products were ligated using overlapping PCR (FIG. 9b—the product is of the expected size).

The overlap PCR product was gel purified and ligated into a sequencing vector for sequencing analysis. This involved transforming bacteria with the sequencing vector containing the ferritin heavy and light chain overlapped genes. The transformed bacteria were then spread on an antibiotic containing plate to separate clones. The cells were incubated overnight to allow colonies to form. Individual colonies were then picked from the plate and grown in liquid media. The plasmids from each clone were isolated and analysed using PCR (FIG. 9c). Clone 4 was found to contain the expected sequence. The DNA from this clone was therefore subsequently used in all further work.

The variable heavy and light chain genes for a murine anti human fibronectin antibody were PCR amplified from a monoclonal hybridoma. These genes have previously been joined by a flexible linker region to form a scFv. This scFv gene fusion was amplified using PCR. The DNA gel of this amplification can be seen in FIG. 10a alongside the ferritin polygene overlap product. The relevant bands were excised from the gel and the DNA purified. This was then used in a further overlap PCR to conjugate the scFv and ferritin polygene (FIG. 10b). The arrowed band is of the expected size for the scFv:ferritin fusion. This was excised and the DNA purified for further use.

The primers used to do this contained sequences to allow for endonuclease (enzymes able to cut specific sequences of double stranded DNA) restriction of the DNA for ligation into a plasmid.

After gel purification, the scFv:ferritin PCR product was restricted using the restriction enzymes (endonucleases) Bam H1 and EcoRI. The purified restricted products were subsequently cloned into two expression vectors; pRSET and pET26b. Clones were isolated as before and the results of a PCR to identify positive clones can be seen in FIG. 11.

Colonies 3-5 and 7 from the set containing the plasmid pRSET and colony 6 from the set containing the plasmid pET26b were selected for sequence analysis.

The resulting data demonstrated that clones pRSET 4 and 5 and pET26b clone 6 contained the scFv:ferritin construct. The clone pRSET 4 was used for protein expression.

Anti-Fibronectin scFv:Ferritin Fusion Protein Expression

To validate the expression of the fusion protein, three 5 ml cultures were grown in LB broth (Luria-Bertani broth: 10 g tryptone, 5 g yeast extract, 10 g NaCl per litre). The cells were induced to express protein using IPTG (isopropyl β-D-1-thiogalactopyranoside) at varying times. The cultures were then lysed in 8M urea and analysed using SDS-PAGE. The gels were stained using Coomassie blue for protein content (results in FIG. 12). Western blots using an anti-polyhistidine antibody were performed to specifically identify the fusion protein (FIG. 12).

The time-points for induction were 2, 3 and 4 hours after inoculation.

The bands seen in the blot demonstrated that the fusion protein was being expressed and could be detected using an anti-histidine antibody. The polypeptide was approximately 75-85 kDa in size. The expression yields were relatively high and over-expression was evident as the fusion protein bands correspond to the very dark bands seen in the Coomassie blue stained gel. Inducing 3 hours after inoculation gave relatively high levels of expression and was used for subsequent expression.

### Generation of the Anti-Fibronectin:MT2 Fusion Protein

### Background

Metallothioneins are intracellular, low molecular weight, cysteine-rich proteins. These proteins are found in all eukaryotes and have potent metal-binding and redox capabilities. MT-1 and MT-2 are rapidly induced in the liver by a variety of metals, drugs and inflammatory mediators. MT2 binds seven divalent transition metals via two metal binding clusters at the carboxyl (α-domain) and amino (β-domain) terminals. Twenty cysteine residues are involved in the binding process.

The sequence of MT2 is:

(SEQ ID No: 5)
MDPNCSCAAGDSCTCAGSCKCKECKCTSCKKSCCSCCPVGCAKCAQ
GCICKGASDKCSCCAPGSAGGSGGDSMAEVQLLE

Together with the anti-fibronectin scFv amino acid sequences, the predicted sequence of a single polypeptide of the fusion protein is (with the linker sequences between the heavy and light antibody genes and between the antibody light chain and MT2 heavy chain highlighted in lower case):

(SEQ ID No: 2)
LVQPGGSLRLSCAASGFTFSSFSMSWVRQAPGKGLEWVSSISGSSGT
TYYADSVKGRFTISRDNSKNTLYLQMNSLRAEDTAVYYCAKPFPYFD
YWGQGTLVTVSSGDgssggsggASTGEIVLTQSPGTLSLSPGERATL
SCRASQSVSSSFLAWYQQKPGQAPRLLIYYASSRATGIPDRFSGSGS
GTDFTLTISRLEPEDFAVYYCQQTGRIPPTFGQGTKVEIKsgggMDP
NCSCAAGDSCTCAGSCKCKECKCTSCKKSCCSCCPVGCAKCAQGCIC
KGASDKCSCCAPGSAGGSGGDSMAEVQLLE

### Assembly of the Anti-Fibronectin:MT2 Fusion Protein Genes

The metallothionein II genes were amplified from a human liver cDNA library using PCR (FIG. 13). The PCR products were of the expected size (˜200 bp).

The PCR product was restricted using the Bgl II restriction enzyme and ligated into a previously cut plasmid (Factor Xa vector).

Colony PCR of selected clones revealed bands for all clones selected (FIG. 14). Clones 2, 4 and 9 were selected for sequencing analysis. Clone 9 was used in further work.

Anti-Fibronectin scFv:MT2 Fusion Protein Expression

To validate expression of the scFv:MT2 fusion protein, three 5 ml cultures were grown in LB broth induced (IPTG) at different time-points as with the ferritin fusion protein. The cultures were lysed in 8M urea and analysed using SDS-PAGE gels stained with Coomassie blue and blotted using an anti-histidine antibody (FIG. 15). Cells induced 4 hours after inoculation produced slightly more protein (lane 3 on both gels). These growth conditions were used for subsequent protein expression.

### Purification of Fusion Proteins

The isolation of soluble protein by isolating, washing and re-solubilising inclusion bodies was employed.

The protocol takes approximately one week to complete. Photographs of a Coomassie blue stained gel and western blot of the re-solubilised scFv:ferritin and scFv:MT2 fusion proteins can be seen in FIG. 16. The fusion proteins are circled—ferritin is in lane 2 on both gels and MT2 is in lane 3 of both gels. A protein molecular weight ladder is in lane 1.

From this, it can be seen that the fusion proteins were successfully expressed and concentrated. These proteins were be used in magnetising protocols and further experiments.

### Example 3

### SPR Analysis

Anti-fibronectin ferritin and MT2 fusion protein inclusion body preparations were used in surface plasmon resonance (SPR) assays using a SensiQ instrument (ICX Nomadics).

For these experiments, a fibronectin peptide was coupled to the surface of a carboxyl chip. The fusion protein preps were then flowed over the chip and association (Kₐ) and dissociation kinetics (Kₐ) determined.

Fusion Protein Samples for Analysis Six samples of each fusion protein, with varying concentration from 0.0013-0.133 μM were produced in running buffer as set out in Table 2 and Table 3 below.

TABLE 2
Metallothionein Fusion Protein 75 kDa:
40 μl 100 μg/ml 75 kDa/360 μl running buffer to give
400 μl 10 μg/ml (0.133 μM) then:

| | | | μl of |
|---|---|---|---|
| μM FP | μg/ml FP | μl of 10 μg/ml FP | running buffer |
| 0.0013 | 0.1 | 20 (of 1 μg/ml) | 180 |
| 0.0065 | 0.5 | 10 | 190 |
| 0.013 | 1 | 20 | 180 |
| 0.05 | 3.75 | 75 | 125 |
| 0.1 | 7.5 | 150 | 50 |
| 0.133 | 10 | 400 | 0 |

TABLE 3
Ferritin ED-B Fusion Protein 270 kDa:
144 μl 100 μg/ml 270 kDa/256 μl running buffer to give
400 μl 36 μg/ml (0.133 μM) then:

| | | | μl of |
|---|---|---|---|
| μM FP | μg/ml FP | μl of 36 μg/ml FP | running buffer |
| 0.0013 | 0.36 | 20 (of 3.6 μg/ml) | 180 |
| 0.0065 | 1.8 | 10 | 190 |
| 0.013 | 3.6 | 20 | 180 |
| 0.05 | 9 | 75 | 125 |
| 0.1 | 18 | 150 | 50 |
| 0.133 | 36 | 400 | 0 |

### Metallothionein

### Sample (Cycles 1-6)=20 μl 0.0013-0.133 μM Metallothionein Fusion Protein

Assay run=MAb & Gly assay cycle (as above)

Sensograms from the above cycles were overlaid using the SensiQ Qdat analysis software, and a model fitted to the data to calculate kinetic parameters (Kₐ, K_{d}). The best estimate of the K_{d} was achieved by fitting a model to just the dissociation part of the data. The result is shown in FIG. 17a. This relates to a Kₐ of 0.00503 s⁻¹ to give a K_{d} of 2.289×10⁻⁹ M (Kₐ 2.197×10⁶ M⁻¹s⁻¹)

### Ferritin

### Sample (Cycles 1-6)=20 μl 0.0013-0.133 μM Ferritin Fusion Protein

Assay run=MAb & Gly assay cycle (as above)

Sensograms from the above cycles were overlaid using the SensiQ Qdat analysis software and a model fitted to the data to calculate kinetic parameters (Kₐ, K_{d}). The best estimate of the K_{d} was achieved by fitting a model to just the dissociation part of the data. The result is shown in FIG. 17b. This relates to a K_{d} of 0.00535 s⁻¹ to give a K_{d} of 6.538×10⁻¹⁰ M (Kₐ 8.183×10⁶ M⁻¹s⁻¹).

### Results

From the above experimental data, it was determined that fibronectin extra domain B (aa 16-42) antigen was successfully coated onto the SensiQ chip As expected, both the 75 kDa Metallothionein Fusion Protein and the 270 kDa Ferritin Fusion Protein recognised and bound to the antigen in a specific manner. Kinetic data on the interactions of the fusion proteins with the antigen were estimated and were found to be similar and in the expected range for both fusion proteins i.e. K_{d}s in the 10⁻⁹ M range compared to 10⁻⁸ M to 10⁻¹⁰ M for most antibody/antigen interactions.

Thus, the values obtained using this instrument suggest binding affinities which compare favourably with the binding affinities of relatively high affinity antibodies. In addition, the data obtained suggest that the fusion proteins have multiple binding sites for antigen. This was expected for the ferritin fusion protein. However, this was not expected for the MT2 fusion protein and would suggest that the fusion protein is forming dimers or higher order multimeric proteins which would increase the avidity of binding.

### Example 4

### Magnetising Ferritin

Ferritin normally contains hydrated iron (III) oxide. In order to produce paramagnetic ferritin, these ions were replaced with magnetite (Fe₃O₄) which has stronger magnetic properties. The method used for this experiment involved the addition to apoferritin of iron ions and oxidation of these ions under controlled conditions.

### Materials

Reverse osmosis water (RO water)
50 mM N-(1,1-Dimethyl-2-hydroxyethyl)-3-amino-2-hydroxypropanesulfonic acid (AMPSO) buffer pH8.6 (Sigma A6659)
0.1M Sodium acetate buffer pH4.5
Phosphate buffered saline (PBS) 10 mM phosphate, 140 mM NaCl pH 7.4
Trimethylamine-N-oxide (TMA) (Sigma 317594)
0.1M ammonium iron (II) sulphate
Horse spleen apoferritin (Sigma A3641)

### Method

Trimethylamine-N-oxide (TMA) was heated in an oven to 80° C. for 30 minutes to remove Me₃N before cooling to room temperature. 114 mg TMA was added to 15 ml RO water to produce a 0.07M solution. The iron and TMA solutions were purged with N₂ for 15 minutes before use.

AMPSO buffer (1 litre) was de-aerated with N₂ for an hour. 3.0 ml apoferritin (66 mg/ml) was added to the AMPSO buffer and the solution de-aerated for a further 30 minutes. The AMPSO/apoferritin solution in a 1 litre vessel was placed into a preheated 65° C. water bath. The N₂ supply line was removed from within the solution and suspended above the surface of the solution to keep the solution under anaerobic conditions. The initial addition of iron ammonium sulphate scavenges any residual oxygen ions that may be in the solution.

Aliquots of the 0.1M iron ammonium sulphate and TMA buffers were added every 15 minutes as follows:

1^{st} addition 600 μl 0.1M iron ammonium sulphate
2^{nd} addition 600 μl 0.1M iron ammonium sulphate and 400 μl TMA
3^{rd} addition 600 μl 0.1M iron ammonium sulphate and 400 μl TMA
4^{th} addition 600 μl 0.1M iron ammonium sulphate and 400 μl TMA
5^{th} addition 900 μl 0.1M iron ammonium sulphate and 600 μl TMA
6^{th} addition 900 μl 0.1M iron ammonium sulphate and 600 μl TMA
7^{th} addition 900 μl 0.1M iron ammonium sulphate and 600 μl TMA
8^{th} addition 900 μl 0.1M iron ammonium sulphate and 600 μl TMA

Upon the latter additions of Fe and TMA, the solution colour changed from a straw colour to dark brown with dark particulates dispersed throughout. This solution is termed “magnetoferritin” from this point onwards.

The magnetoferritin solution was incubated at room temperature overnight with a strong neodymium ring magnet held against the bottle. The following day, dark solid material had been drawn towards the magnet as can be seen in the photographs in FIG. 18.

### Concentration of Magnetoferritin

Five hundred millilitres of the magnetoferritin solution was passed through 5 Macs® LS columns on magnets (with approximately 100 ml magnetoferritin passing through each column) The solution which flowed through the columns (termed ‘flow-through’) was collected in Duran bottles. The captured material from each column was eluted using 3 ml PBS by removing the columns from the magnets, adding the 3 mls PBS and using the supplied plunger resulting in approximately 4.5 ml from each column. Approximately 1 ml was stored at 2-8° C. for later analysis (termed ‘pre-dialysis concentrated magnetoferritin’). The remainder of the eluted solution (˜20 ml) was dialysed (termed ‘post-dialysis concentrated magnetoferritin’) against 5 litres of PBS at 4° C. overnight to remove excess Fe and TMA. The change in colour of the solution was noted. The original magnetoferritin was dark brown, the flow through straw coloured and the Macs® column concentrated material dark brown to black.

Dialysis tubing (Medicell International Ltd. Molecular weight cut-off 12-14000 Daltons ˜15 cm) was incubated in RO water for ten minutes to soften the tubing. The magnetically isolated concentrated magnetoferritin was transferred to the dialysis tube and incubated in 5 litres PBS at 2-8° C. with stirring overnight. The PBS solution was refreshed three times the following day at two hour intervals with dialysis continuing at 2-8° C.

### Analysis of Magnetoferritin

In order to compare the amount of magnetic protein isolated using the magnet, enzyme linked immunosorbant assay (ELISA) analysis was performed.

### Materials

Carbonate Buffer (0.159 g sodium carbonate and 0.3 g sodium bicarbonate in 100 mls RO water).
Phosphate buffered saline (PBS) 10 mM phosphate, 140 mM NaCl pH 7.4
1% bovine serum albumin (BSA (Celliance 82-045-2)) in PBS
Horse spleen apoferritin (Sigma Aldrich A3641)
Rabbit anti-horse ferritin antibody (Sigma Aldrich F6136)
Goat anti-rabbit antibody (Sigma A3687)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

Dilutions of apoferritin were made (50 μg/ml, 25 μg/ml, 12.5 μg/ml, 6.25 μg/ml, 3.125 μg/ml and 1.5625 μg/ml) for quantification of the magnetoferritin.

The magnetoferritin (unpurified), concentrated pre-dialysis, post-dialysis and flow through was diluted in carbonate buffer at the following dilutions:

Magnetoferritin, pre-dialysis and post-dialysis dilutions:

100, 200, 400, 800, 1600, 3200, 6400 and 12800 fold dilution.

Flow-through:

10, 20, 40, 80, 160, 320, 640 and 1280 fold dilution.

100 μl of each solution was added to wells of a microtitre plate in duplicate. Carbonate buffer (100 μl) was added to two wells as a negative control. The plate was incubated overnight at 4° C. The next day, the solution was flicked off and the wells blocked using 200 μl 1% BSA at room temperature for an hour. After washing three times with 300 μl PBS per well, the wells were patted dry before the addition of 100 μl 10 μg/ml anti-horse ferritin antibody. This was incubated for an hour at room temperature before being removed and wells washed as before.

AP-conjugated anti rabbit antibody was diluted 1 in 3500 in PBS to give a concentration of 7.43 μg/ml and incubated at room temperature for an hour. The antibody conjugate was removed and wells washed as before. AP substrate (100 μl) was added to each well and allowed to develop for 15 minutes before the addition of stop solution. Absorbances were recorded using a Varioskan Flash instrument (Thermo Fisher).

The Macs® columns retained over 35 times the amount of magnetoferritin found in the flow through indicating that magnetisation of the protein had been successful.

### Production of Apoferritin/Demineralisation of Horse Spleen Ferritin.

### Materials

0.1M sodium acetate buffer pH 4.5
Thioglycolic acid (Sigma T6750)
Horse Spleen Ferritin (Sigma 96701)
Phosphate buffered saline (PBS) 10 mM phosphate, 140 mM NaCl pH 7.4

### Method

Dialysis tubing was softened in RO water for 10 minutes. 10 ml 0.1M sodium acetate buffer was added to 1 ml Horse Spleen Ferritin (125 mg/ml) in the dialysis tubing which was clipped at both ends. The dialysis bag was transferred to 0.1M sodium acetate buffer (˜800 ml) which had been purged with N₂ for one hour. Thioglycolic acid (2 ml) was added to the buffer and N₂ purging was continued for two hours. A further 1 ml thioglycolic acid was added to the sodium acetate buffer followed by another thirty minutes of N₂ purging. The sodium acetate buffer (800 ml) was refreshed and purging continued. The demineralisation procedure was repeated until the ferritin solution was colourless. The N₂ purge was stopped and the apoferritin solution was dialysed against PBS (2 L) for 1 h with stirring. The PBS was refreshed (3 litres) and the apoferritin solution was dialysed in PBS at 2-8° C. overnight.

### Results

The ferritin solution changed colour during the procedure from light brown to colourless indicating removal of iron.

### Analysis of Heat Treatment on the Anti-Fibronectin:Ferritin Fusion Protein

### Materials

Carbonate buffer (0.159 g sodium carbonate, 03 g sodium bicarbonate in 100 mls water)
Phosphate buffered saline (PBS)
1% bovine serum albumin (BSA (Celliance 82-045-2)) in PBS
Fibronectin peptide
Anti-fibronectin: ferritin fusion protein (scFv:ferritin)
Anti-human ferritin murine monoclonal antibody (Santa Cruz SC51887)
Anti-mouse alkaline phosphatase antibody (Sigma A3562)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

100 μl (at 100 μg/ml) scFv:ferritin was transferred to a thin walled PCR tube and heated in a thermocycler at 60° C. for 30 minutes.

Wells of a microtitre plate were coated with fibronectin peptide (supplied at 1.5 mg/ml) diluted in carbonate buffer to 15 μg/ml and incubated overnight at 4° C. Excess solution was flicked off and the plate blocked using 1% BSA in PBS for 1 hour at room temperature. This was flicked off and the plate washed three times using PBS. The scFv:ferritin fusion protein and heat treated scFv:ferritin fusion protein were added to wells at a concentration of 33 μg/ml (100 μl each). The ferritin fusion proteins were incubated for 2 hours at room temperature before being removed and the wells washed as before. Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted (50 μl+950 μl PBS) and added at a volume of 100 μl to all wells. This was incubated at room temperature for an hour and removed as before. Substrate was added to all wells and incubated at room temperature for 45 minutes and the reaction stopped using stop buffer. Absorbances were recorded using a Varioskan Flash instrument (Thermo Fisher Electron).

The scFv:ferritin retains binding ability to fibronectin and remains detectable by the anti-human ferritin monoclonal antibody after heating to 60° C. for 30 minutes (FIG. 20).

### Anti Fibronectin:Ferritin Fusion Protein Demineralisation

### Materials

Anti-fibronectin: ferritin fusion protein (scFv:ferritin).
0.1M sodium acetate buffer
Thioglycolic acid (70% w/w Sigma T6750)
Phosphate buffered saline (PBS) 10 mM phosphate, 140 mM NaCl pH 7.4

### Method

The scFv:ferritin fusion protein was thawed from −20° C. to room temperature. Nine millilitres of 100 μg/ml was dispensed into softened dialysis tubing. The tubes which had contained the fusion protein were rinsed with a total of 1 ml sodium acetate buffer which was added to the 9 ml of protein (to give a 0.9 mg/ml solution). 800 ml sodium acetate buffer was purged with N₂ for 15 minutes before the dialysis bag was added. The solution was then purged for a further 2 hours. 2 ml thioglycolic acid was added to the buffer which continued to be purged using N₂. After a further 2 hours, another 1 ml of thioglycolic acid was added. The buffer was refreshed (800 ml pre-purged sodium acetate buffer containing 3 ml thioglycolic acid) and dialysis continued under N₂ for 1 hour. The dialysis bag was then transferred to 2 litres PBS at room temp (no N₂) then overnight at 4° C. in 3 litres PBS. This demineralised fusion protein was then used to produce paramagnetic fusion protein by the addition of iron and controlled oxidation as below.

Production of Magnetic scFv:Ferritin

### Materials

Reverse osmosis water (RO water)
50 mM N-(1,1-Dimethyl-2-hydroxyethyl)-3-amino-2-hydroxypropanesulfonic acid (AMPSO) buffer pH8.6 (Sigma A6659)
0.1M Sodium acetate buffer pH4.5
Phosphate buffered saline (PBS) 10 mM phosphate, 140 mM NaCl pH 7.4
Trimethylamine-N-oxide (TMA) (Sigma 317594)
0.1M ammonium iron (II) sulphate

Trimethylamine-N-oxide (TMA) was heated in an oven to 80° C. for 30 minutes to remove Me₃N before cooling to room temperature. 114 mg TMA was added to 15 ml RO water to produce a 0.07M solution. The iron and TMA solutions were purged with N₂ for 15 minutes before use.

The demineralised fusion protein contained within a dialysis bag (detailed above) was dialysed against 1 litre AMPSO buffer for 2 hour at room temp with stirring under nitrogen. The demineralised scFv:ferritin (˜10 ml) was transferred to a conical flask. 18 μl iron solution was added to the demineralised protein solution whilst purging with N₂ to scavenge any residual oxygen. After 25 minutes, 15 μl iron and 10 μl TMA were added.

The following further amounts of iron and TMA buffers were then added at 15 minute intervals:

3^{rd} addition: 30 μl iron+20 μl TMA.
4^{th} addition: 15 μl iron+10 μl TMA
5^{th} addition: 15 μl iron+10 μl TMA
6^{th} addition: 15 μl iron+10 μl TMA

The magnetised protein was passed through a Macs® LS column. The flow through was passed though a second time to try and increase capture efficiency. The magnetised protein was eluted from the column by removing the column from the magnet and adding 1 ml PBS and using the plunger (eluate approx 2 ml). This represents a two-fold dilution of the protein on the column.

Eluted protein and controls were coated onto a microtitre plate for analysis as detailed below.

Analysis of the scFv:Magnetoferritin Fusion Protein by ELISA

In order to ascertain if the magnetised fusion protein retains binding to an anti-ferritin monoclonal antibody an enzyme linked immunosorbant assay was performed.

### Materials

Carbonate buffer (0.159 g sodium carbonate, 03 g sodium bicarbonate in 100 mls water) pH 9.6
Phosphate buffered saline (PBS) 10 mM phosphate, 140 mM NaCl pH 7.4
Fibronectin peptide
Anti-fibronectin:ferritin fusion protein (scFv:ferritin)
Anti-human ferritin murine monoclonal antibody (Santa Cruz SC51887)
Anti-mouse alkaline phosphatase antibody (Sigma A3562)
Substrate liquid stable phenolphthalein phosphate
Stop Solution (212 g sodium carbonate, 110.5 g 3-(cyclohexylamino)-1-propanesulfonic acid (CAPS), 217 g ethylenediamine tetraacetic acid (EDTA) 80 g sodium hydroxide, water to 5 litres)
Maxisorb Microtitre plate (NUNC Cat: 468667)

### Method

### Fusion Protein Coated Wells

Wells were coated with scFv:ferritin (untouched), scFv:magnetoferritin, scFv:magnetoferritin eluted from the Macs® column and the flow through at a concentration of 1 in 3 in carbonate buffer. The plate was incubated over a weekend at 4° C. Excess solution was flicked off and the plate blocked using 1% BSA in PBS for 1 hour at room temperature. This was flicked off and the plate washed three times using PBS (300 μl/well for each wash). Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted to 10 μg/ml and added at a volume of 100 μl to all wells. This was incubated at room temperature for an hour and removed as before. Substrate was added to all wells and incubated at room temperature for an hour and the reaction stopped using stop buffer. Absorbances were recorded using a Varioskan Flash instrument (Thermo Fisher Electron) (see FIG. 21a).

### Fibronectin Coated Wells

Wells of a microtitre plate were coated with 100 μl fibronectin peptide (supplied at 1.5 mg/ml) diluted in carbonate buffer to 15 μg/ml. The plate was incubated overnight at 2-8° C. Excess solution was flicked off and the wells washed three times in 300 μl PBS. The scFv:ferritin fusion proteins were added neat to the appropriate wells (100 μl) in duplicate. The plate was then incubated for an hour at room temperature. The solution was flicked off and the wells washed three times in 300 μl PBS. Mouse anti-ferritin antibody was added at a concentration of 20 μg/ml and added at a volume of 100 μl to each well and incubated at room temperature for an hour. This was removed and the wells washed as before. Goat anti-mouse AP conjugated antibody was diluted to 10 μg/ml and added at a volume of 100 μl to all wells. This was incubated at room temperature for an hour and removed as before. Substrate was added to all wells and incubated at room temperature for 45 minutes and the reaction stopped using stop buffer. Absorbances were recorded using a Varioskan Flash instrument (Thermo Fisher Electron) (see FIG. 21b).

The Macs® columns have concentrated the magnetised fusion protein and it is still recognised by the monoclonal anti-ferritin antibody, indicating that the anti-fibronectin-ferritin fusion protein has been magnetised and retained structural integrity. The data also indicates that the magnetised anti-fibronectin ferritin fusion protein retains binding ability to its target antigen and thus illustrates a bi-functional single chain fusion protein that is both magnetisable and can bind a target selectively.

### Example 5

### Isolation of Platelets and FACS Analysis

An experiment was conducted to demonstrate the ability of the anti-fibronectin:ferritin fusion protein (scFv:ferritin) to select platelets expressing fibronectin from other cell types.

Plasma from a sample of blood which had been stored in an EDTA vacutainer at 4° C. for three days to allow most cells to settle was exposed to air for 30 minutes to activate platelets. 10 μl of this was mixed with 100 μl magnetised scFv:ferritin as described above. The magnetic fusion protein/plasma mix was incubated at room temperature for 30 minutes (10 μl was retained for analysis) before being passed through a magnetised, pre-equilibrated LS MACS column (Miltenyi Biotec). The flow through was retained for analysis. The bound fraction was eluted from the column using the supplied plunger. The fractions were diluted to 500 μl in PBS and analysed using forward and side scatter by fluorescence activated cell sorting (FACS).

The results are shown in Table 4. It should be recognised that with FACS analysis the sample is analysed until a set number of events (e.g. 10,000) have been recorded. Thus, the volume sampled can vary enormously dependent on the concentration of cells. This is particularly important when one is comparing samples with high cellular concentrations with samples where many of the cells have been removed. When calculating the efficiency of cellular removal or isolation procedure, it is necessary to correct for this change of sample volume. This is done within Table 4.

TABLE 4
Number of FACS cell type related events and selectivity and isolation
efficiency of isolating platelets from plasma.

| | | | Flow | Captured |
|---|---|---|---|---|
| Total Events | 10000 | Plasma | Though | (magnetised) |
| No of non-lymphocytes | | 9819 | 8476 | 9982 |
| No of Lymphocytes | | 181 | 1524 | 18 |
| Non-lymphocyte:lymphocyte | | 54.2 | 5.6 | 554.6 |
| ratio | | | | |
| Relative volume | | 1.0 | 8.4 | 0.1 |
| Selectivity efficiency | 99.7 | | | |
| (non-lymphocytes/ | | | | |
| lymphocytes) | | | | |
| Isolation efficiency (% of | 90.1 | | | |
| platelets captured) | | | | |

It can be seen that 90% of available platelets were captured with this un-optimised procedure with selectivity over lymphocytes of almost 100%. This demonstrates the ability of the scFv:ferritin protein to bind fibronectin displayed on the surface of platelets.

Visual inspection with a microscope (results not shown) correlated with the FACS analysis showing that fusion protein binds to platelets leading to the formation of large, granular aggregates.

### Example 6

### Further Protocols

Magnetisation of scFv MT2 Fusion Protein

The scFv-MT2 fusion proteins may be magnetised by replacing zinc ions with manganese and cadmium ions. Methods to do this may be optimised as required. The methods to achieve this include the depletion of zinc by dialysis followed by replacement, also using dialysis with adaptations of published protocols if required.

In detail, these protocols are as follows:
1. Dissolve 5 mg MT2 in 5 ml buffer (4.5M urea, 10 mM Tris base, 0.1M dithiothreitol (DTT), 0.1% mannitol and 0.5 mM Pefabloc, pH 11) to strip the protein of the metal ions.
2. Dialyse in the same buffer for 1 hours.
3. Refold the protein by dialysing in buffer 1 (10 mM tris base, 2M urea, 0.1M DTT, 0.1% mannitol, 0.5 uM Pefabloc and 1 mM Cd²⁺/Mn²⁺ pH 11) for 72 hours.
4. Change dialysis buffer to buffer 2 (as above but with urea at a concentration of 1M) and dialyse for 24 hours.
5. Change the dialysis buffer to a buffer as above containing no urea. Dialyse for 24 hours.
6. Change the dialysis buffer as in step 5 to a buffer with pH 8.8. Dialyse for 24 hours.
7. Change the buffer as in step 6 to a buffer containing no mannitol and dialyse as before.
8. Change the buffer as in step 7 to a buffer containing no Cd²⁺/Mn²⁺ and dialyse for 24 hours.

The binding characteristics may be assessed as above in Example 3 for the ferritin fusion protein.

1. A vector for use in delivering a therapy to or removing a therapy from a site within a patient, wherein the vector comprises:
a) a binding moiety; and
b) a therapeutic agent,
wherein the binding moiety comprises a metal-binding protein, polypeptide or peptide which is bound to or encapsulates a magnetic or magnetisable substance, and wherein the vector is to be administered using a device comprising an electromagnet and an element suitable for bringing the therapeutic agent into proximity with the site.

2. A vector for use in delivering a therapy to or removing a therapy from a site within a patient according to claim 1 wherein the element suitable for bringing the therapeutic agent into proximity with the site is a catheter.

3. A vector for use in delivering a therapy to or removing a therapy from a site within a patient according to claim 1 wherein the therapeutic agent is a radio-isotope or a chemotherapeutic agent.

4. A vector for use in delivering a therapy to or removing a therapy from a site within a patient according to claim 1 wherein the therapeutic agent is a thrombolytic or anti-thrombotic agent.

5. A vector for use in delivering a therapy to or removing a therapy from a site within a patient according to claim 4 wherein the therapeutic agent is antithrombin III.

6. A vector for use in delivering a therapy to or removing a therapy from a site within a patient according to claim 1 wherein the therapeutic agent is the magnetic or magnetisable substance and the magnetic or magnetisable substance is suitable for delivering hyperthermic therapy to the site on exposure to an electromagnetic field.

7. A vector for use in delivering a therapy to or removing a therapy from a site within a patient according to claim 1 wherein the vector further comprises a recognition moiety for targeting the vector to the site within the body of the patient.

8. A vector for use in delivering a therapy to or removing a therapy from a site within a patient according to claim 7 wherein the recognition moiety is selected from an antibody or a fragment of an antibody, a receptor or a fragment of a receptor, a protein, a polypeptide, a nucleic acid, and an aptamer.

9. A vector for use in delivering a therapy to or removing a therapy from a site within a patient according to claim 8 wherein the recognition moiety is selected from a variable polypeptide chain of an antibody (Fv), a T-cell receptor or a fragment of a T-cell receptor.

10. A vector for use in delivering a therapy to or removing a therapy from a site within a patient according to claim 9 wherein the recognition moiety is a single chain of a variable portion of an antibody (sc-Fv).

11. A vector for use in delivering a therapy to or removing a therapy from a site within a patient according to claim 1 wherein the binding moiety comprises a protein or a metal-binding domain of a protein selected from lactoferrin, transferrin, ferritin, a ferric binding protein, or a metallothionein (MT).

12. A vector for use in delivering a therapy to or removing a therapy from a site within a patient according to claim 11 wherein the binding moiety comprises ferritin.

13. A vector for use in delivering a therapy to or removing a therapy from a site within a patient according to claim 1 wherein the magnetic or magnetisable substance is a transition and/or lanthanide metal atom and/or ions and/or a compound comprising such ions.

14. A vector for use in delivering a therapy to or removing a therapy from a site within a patient according to claim 13 wherein the transition metal and/or lanthanide ions comprise any one or more ions of Fe, Co, Ni, Mn, Cr, Cu, Zn, Cd, Y, Gd, Dy, or Eu.

15. A vector for use in delivering a therapy to or removing a therapy from a site within a patient according to claim 14 wherein the one or more metal ions comprise any one or more of Fe²⁺, Fe³⁺, Co²⁺, Co³⁺, Mn²⁺, Mn³⁺, Mn⁴⁺, Ni²⁺, Zn²⁺, Gd³⁺ and Cd²⁺.

16. A vector for use in delivering a therapy to or removing a therapy from a site within a patient according to claim 1 further comprising a plurality of moieties for binding or encapsulating the magnetic or magnetisable substance.

17. A pharmaceutical composition comprising:
(a) a binding moiety;
(b) a therapeutic agent; and
(c) a recognition moiety
wherein the binding moiety comprises a metal-binding protein, polypeptide or peptide which is bound to or encapsulates a magnetic or magnetisable substance.

18. A pharmaceutical composition according to claim 17 wherein the therapeutic agent is the magnetic or magnetisable substance and the magnetic or magnetisable substance is suitable for delivering hyperthermic therapy to a site within a patient on exposure to an electromagnetic field.

19. A pharmaceutical composition according to claim 17 further comprising a therapeutic agent selected from a radio-isotope or a chemotherapeutic agent.

20. A pharmaceutical composition according to claim 17, wherein the binding moiety comprises a protein or a metal-binding domain of a protein selected from lactoferrin, transferring, ferritin, a ferric binding protein, frataxin, a siderophore and a metallothionein (MT).

21. A pharmaceutical composition according to claim 17, wherein the magnetic or magnetisable substance is selected from the group consisting of a transition and/or lanthanide metal atom and/or ions and/or a compound comprising such ions.

22. A pharmaceutical composition according to claim 17, wherein the recognition moiety is selected from an antibody or a fragment of an antibody, a receptor or a fragment of a receptor, a protein, a polypeptide, a nucleic acid, and an aptamer.

23. A method of treating diseased cells or tissue susceptible to hyperthermia in a patient comprising administering a pharmaceutical composition of claim 17, wherein the composition is to be administered prior to exposing the patient to an electromagnetic field.

24. (canceled)

25. A method for administering a vector to a site within a patient using a device comprising an electromagnet and an element suitable for bringing the vector into proximity with the site, comprising the steps of:
adhering the vector to the element using the electromagnet;
inserting the element into the body of the patient; and
using the electromagnet to control the release of the vector from the element,
wherein the vector comprises a binding moiety and a therapeutic agent and wherein the binding moiety comprises a metal-binding protein, polypeptide or peptide which is bound to or encapsulates a magnetic or magnetisable substance.

26. A method for removing a vector from a site within a patient using a device
comprising an electromagnet and an element suitable for bringing the vector into proximity with the site, comprising the steps of:
inserting the element into the body of the patient;
adhering the vector to the element using the electromagnet; and
removing the element from the body of the patient,
wherein the vector comprises a binding moiety and a therapeutic agent and wherein the binding moiety comprises a metal-binding protein, polypeptide or peptide which is bound to or encapsulates a magnetic or magnetisable substance.
